(19) United States
(12) Patent Application Publication
(10) Pub. No.: US 2026/0256349 A1
(43) Pub. Date: Sep. 3, 2026

(54) INTRAORAL SCANNER SYSTEM

(71) Applicant: 3SHAPE A/S, Copenhagen K (DK)

(72) Inventors: Scott Nicolai JESPERSEN, Copenhagen K (DK); Kostiantyn KLIUCHEVSKYI, Copenhagen K (DK); Signe Friis SCHACK, Copenhagen K (DK); Astrid Anna TOFTEGAARD, Copenhagen K (DK); Aysen CHRISTENSEN, Copenhagen K (DK)

(73) Assignee: 3SHAPE A/S, Copenhagen K (DK)

(21) Appl. No.: 18/880,076

(22) PCT Filed: Jun. 22, 2023
(86) PCT No.: PCT/EP2023/066977

§ 371 Date: Dec. 30, 2024

(30) Foreign Application Priority Data
Jul. 1, 2022 (EP) 22182610.0

(51) Int. Cl. A61B 1/24; A61B 1/00; A61B 1/04; A61B 90/00

(52) U.S. Cl. CPC A61B 1/24; A61B 1/00135; A61B 1/04; A61B 90/08; A61B 2090/0808

(57) ABSTRACT

An intraoral scanner system includes an intraoral scanner configured to acquire intraoral scan data from a three-dimensional dental object during a scanning session. The intraoral scanner includes a protection tip and a main body. The protection tip is detachable to the main body. The intraoral scanner system also includes a sleeve configured to cover at least a part of the main body of the intraoral scanner.

### TECHNICAL FIELD

Generally, the present disclosure relates to an intraoral scanner system, and more particularly relates to an intraoral scanner system having an intraoral scanner and a sleeve partially covering the intraoral scanner.

### BACKGROUND

Intraoral scan devices are, generally, used by dentists to capture a direct optical impression of patient's teeth. Intraoral scan devices, generally, include a body portion and a tip portion that is detachably attached to the body portion. Sometimes, after use of the intraoral scan device, a fluid may enter the body portion though a small opening that may exist between the body portion and the tip portion, which is not desirable. Moreover, there are chances of intraoral scan device getting dirty after prolong use. Also, outer surface of the intraoral scan device may get scratched during handling of the device, which is also undesirable.

### SUMMARY

One aspect of the disclosure is to provide a sleeve for covering an intraoral scanner.

One aspect of the disclosure is an intraoral scanner system having an intraoral scanner and a sleeve arranged covering the intraoral scanner.

One aspect of the disclosure is to provide a sleeve that may form a sealing connection between the intraoral scanner to prevent a flow of fluid between the sleeve and the intraoral scanner.

One aspect of the disclosure is to provide a sleeve that may allow a flow air between a battery compartment arranged at a rear of the intraoral scanner and an ambient to enable a removal of heat from the battery compartment.

One aspect of the disclosure is to provide a sleeve that may be easily assembled over the intraoral scanner.

These aspects are provided by an intraoral scanner system that may include an intraoral scanner and a sleeve. The intraoral scanner may be configured to acquire intraoral scan data from a three-dimensional dental object during a scanning session. The intraoral scanner may include a main body and protection tip that may be detachable to the main body. The sleeve may be configured to cover at least a part of the main body of the intraoral scanner.

The sleeve may comprise a shape that follows at least a part of an outer surface of the protection tip and the main body such that the shape of the sleeve may be configured to tighten around at least a part of the protection tip, when the handheld intraoral scanner is slided into the sleeve.

The protection tip may have a trumpet shaped end which may be configured to snap couple to the main body. The circumference of the protection tip may increase gradually from the tip towards the end of the protection tip that may be detachably mounted to the main body. The circumference of the protection tip may increase more rapidly at the trumpet shaped end. The increase of the circumference may result in that the inner dimension of the protection tip increases. The at least part of the protection tip may include the trumpet shaped end, and the sleeve may be configured to slight onto the trumpet shape end, and the material of the sleeve may expand until the flexibility of the material may prevent it from expanding. The material may have some elasticity that may be configured to shrink, and this shrinking behavior may tight the sleeve onto the protection tip, and more precisely, onto the trumpet shaped end of the protection tip.

The sleeve may comprise an indicator that may be configured to guide how the intraoral scanner should be accommodated by the sleeve.

The sleeve may comprise an indicator that may be configured to indicate that the sleeve is drawn correctly along the main body.

The sleeve may comprise a first end and a second end opposite to the first end, a top part and a bottom part, wherein the top part and the bottom part may be configured to be aligned with a top part of the main body and a bottom part of the main body respectively, and wherein the sleeve may comprise the indicator on the top part that may be configured to visually guide a user how the intraoral scanner should be accommodated by the sleeve.

According to some aspects, wherein the indicator may be a visual marking. The visual marking may be adapted to be arranged over a user interface of the intraoral scanner. This feature allows a correct/right positioning and assembly of the sleeve over the intraoral scanner.

The indicator may be an uneven surface which has to match a similar uneven surface of the intraoral scanner when arranging the sleeve over the intraoral scanner. Thus, the indicator may comprise an elevated part.

The indicator may be configured to mimic the shape of the user interface to indicate a user that sleeve may be positioned over the scanner such that the indicator may be arranged covering the user interface.

The sleeve may comprise a first end and a second end arranged opposite to the first end. A first opening of the sleeve may be arranged at the first end and a second opening of the sleeve may be arranged at the second end. Two oppositely positioned openings may allow an easy insertion of the sleeve over the scanner.

In an aspect, the second opening may be larger than the first opening. This feature allows for an easy insertion of the scanner inside the sleeve from the protection tip side An outer surface of a housing of the intraoral scanner or a tip of the intraoral scanner may be partly curved shaped along a transverse axis and/or a longitudinal axis of the housing, and that demands a certain shape of at least an opening of the sleeve for obtaining an optimal sealing. The first opening may be at least partly made from a curved cut to obtain an optimal sealing to the curved shaped housing. This feature enables a good sealing between the sleeve and the intraoral scanner and prevents a flow of fluid between the sleeve and the intraoral scanner through the first opening.

A circumference of the first opening may be curved along a transverse axis of the sleeve. This feature enables a good sealing between the sleeve and the intraoral scanner and prevents a flow of fluid between the sleeve and the intraoral scanner through the first opening.

The second opening may be configured to allow airflow from a handheld intraoral scanner towards ambient air to remove heat from a battery compartment of the intraoral scanner. A part of the second opening may be curved along a transverse axis of the sleeve. The part is attached to the housing of the intraoral scanner via an adhesive material.

According to some aspects, at least a part of the sleeve may include a shape that corresponds to at least a part of an outer shape of the protection tip or to at least a part of an outer shape of the main body of the intraoral scanner. This feature allows for a tight fit between the sleeve and the intraoral scanner.

The sleeve may be configured to accommodate the intraoral scanner and may be configured to provide space for the intraoral scanner to slide into the sleeve.

A cross-sectional area of the intraoral scanner may vary along a longitudinal axis, and a cross-sectional area of the scanner may be smaller than a cross-sectional area of the sleeve along a first part of the scanner along the longitudinal axis, and a cross-sectional area of the scanner may be larger than a cross-sectional area of the sleeve along a second part of the scanner along the longitudinal axis. These features allow for an easy assembly of the sleeve over the scanner.

The sleeve may comprise a transition area along the longitudinal axis. At a first part of the transition area, the cross-sectional area of the scanner may be equal to the cross-sectional area of the sleeve, and at a second part of the transition area, the cross-sectional area of the scanner may be larger than the cross-sectional area of the sleeve providing a sealing effect between the scanner and sleeve.

The first opening may be smaller than the cross-sectional area of at least a part of the intraoral scanner or the cross-sectional area of at least a part of the protection tip of the intraoral scanner. This feature allows that a sealing connection is formed between the sleeve and the intraoral scanner at the location of the first opening.

The sleeve may comprise a top part and a bottom part.

The top part may be arranged opposite to the bottom part.

When an intraoral scanner is arranged within the sleeve, the top part may be arranged at a user interface of the scanner.

A circumference of the first opening may be curved at least between the bottom part and the upper part.

The top part of the sleeve may be longer than the bottom part of the sleeve to allow a passage of air between the battery compartment and the ambient when the sleeve is arranged over the scanner and scanner is being operated.

At the second end, the top part may be longer than the bottom part providing an air gap between the intraoral scanner and the sleeve when the intraoral scanner may be arranged within the sleeve. This feature prevents a closure of the air gap during operation of the intraoral scanner.

The bottom part of the sleeve may comprise a first bottom distal end and a second bottom distal end opposite to the first bottom distal end. The top part of the sleeve may comprise a first top distal end and a second top distal end opposite to the first top distal end. The top part of the sleeve may be longer than the bottom part of the sleeve. A part of the first top distal end of the sleeve and a part of the first bottom distal end of the sleeve may meet at the first end of the sleeve.

Since only a part of the first top distal end of the sleeve and only a part of the first bottom distal end of the sleeve may meet at the first end of the sleeve, a first opening of the sleeve may be provided.

A part of a second bottom distal end of the sleeve may meet with a part of the top part of the sleeve located between the first top distal end of the sleeve and the second top distal end of the sleeve.

Since only a part of the second bottom distal end of the sleeve may meet with only a part of the top part of the sleeve located between the first top distal end of the sleeve and the second top distal end of the sleeve, a second opening of the sleeve may be provided, that is opposite to the first opening of the sleeve. Since the bottom part of the sleeve is shorter than the top part of the sleeve, the second opening may allow for an air gap at the rear of the intraoral scanner, configured to allow an air flow to and from the intraoral scanner, that is configured to cool the intraoral scanner.

The indicator may be arranged on the top part of the sleeve.

The sleeve may be to provide hermetic sealing of a surface of at least a part of the intraoral scanner and/or of a surface of at least a part of a detachable tip of a handheld intraoral scanner.

The sleeve may be configured to seal at least a part of a surface of the protection tip and/or at least a part of the main body to prevent a flow fluid between the sleeve and the intraoral scanner. The shape of the first opening of the sleeve may be adapted to the shape of an outer surface of the protection tip. For example, the protection may be curved shaped along an axis being parallel to the transverse axis of the housing of the intraoral scanner.

The housing may be similar to the main body.

The sleeve may be configured to prevent fluid from passing through between the first opening and at least a part of a surface of the protection tip and/or at least a part of a surface of the main body.

The sleeve may at least partly be made of a flexible material to allow an easy assembly of the sleeve over the intraoral scanner.

The sleeve may be at least partly made of a first material that is more flexible than a second material of the main body of the intraoral scanner.

The sleeve may at least partly be made of a polymer material.

The sleeve may at least partly be made of a biomaterial or a biodegradable material.

The sleeve may comprise an adhesive material, configured to provide an adhesive connection between the sleeve and the intraoral scanner to securely attach the sleeve with the intraoral scanner.

The adhesive material may be covered by a tape that may be configured to cover the adhesive material to prevent deterring or damage of the adhesive material.

The adhesive material may be provided at the second end or at a second top distal end of the sleeve opposite to the first top distal end of the sleeve. Thereby, the adhesive material fixates the sleeve onto the main body preventing the sleeve from sliding off the main body during use or at least sliding away from the optimal position of the sleeve onto the main body. The optimal sealing of the intraoral scanner is maintained during a scanning session. Furthermore, the arrangement of the adhesive material allows air to flow into the main body for cooling the electronics within the main body.

The adhesive may be arranged at the top part of the sleeve, and closer to the second end than to the first end.

### BRIEF DESCRIPTION OF THE ACCOMPANYING DRAWINGS

Aspects of the disclosure may be best understood from the following detailed description taken in conjunction with the accompanying figures. The figures are schematic and simplified for clarity, and they just show details to improve the understanding of the claims, while other details are left out. Throughout, the same reference numerals are used for identical or corresponding parts. The individual features of each aspect may each be combined with any or all features of the other aspects. These and other aspects, features and/or technical effect will be apparent from and elucidated with reference to the illustrations described hereinafter in which:

FIG. 1 illustrates a top view of an intraoral scanner system having an intraoral scanner and a sleeve arranged over the intraoral scanner, in accordance with an embodiment of the disclosure;

FIG. 2 illustrates a side view of the intraoral scanner system of FIG. 1, in accordance with an embodiment of the disclosure;

FIG. 3 illustrates a top view of the intraoral scanner, in accordance with an embodiment of the disclosure;

FIG. 4 illustrates a top perspective view of the sleeve, in accordance with an embodiment of the disclosure; and

FIG. 5 illustrates a perspective view of the sleeve of FIG. 4 with a top part of the sleeve disengaged from a bottom part of the sleeve, in accordance with an embodiment of the disclosure.

### DETAILED DESCRIPTION

The detailed description set forth below in connection with the appended drawings is intended as a description of various configurations. The detailed description includes specific details for the purpose of providing a thorough understanding of various concepts. However, it will be apparent to those skilled in the art that these concepts may be practiced without these specific details. Several aspects of the devices, systems, mediums, programs and methods are described by various blocks, functional units, modules, components, circuits, steps, processes, algorithms, etc. (collectively referred to as “elements”). Depending upon particular application, design constraints or other reasons, these elements may be implemented using electronic hardware, computer program, or any combination thereof.

The electronic hardware may include microprocessors, microcontrollers, digital signal processors (DSPs), field programmable gate arrays (FPGAs), programmable logic devices (PLDs), gated logic, discrete hardware circuits, and other suitable hardware configured to perform the various functionality described throughout this disclosure. Computer program shall be construed broadly to mean instructions, instruction sets, code, code segments, program code, programs, subprograms, software modules, applications, software applications, software packages, routines, subroutines, objects, executables, threads of execution, procedures, functions, etc., whether referred to as software, firmware, middleware, microcode, hardware description language, or otherwise.

A scanning for providing intraoral scan data may be performed by a dental scanning system that may include an intraoral scanner such as the TRIOS series scanners from 3Shape A/S. The dental scanning system may include a wireless capability as provided by a wireless network unit. The scanning device may employ a scanning principle such as triangulation-based scanning, confocal scanning, focus scanning, ultrasound scanning, x-ray scanning, stereo vision, structure from motion, optical coherent tomography OCT, or any other scanning principle. Another example of a scanning device is a triangulation scanner, where a time varying pattern is projected onto the dental object and a sequence of images of the different pattern configurations are acquired by one or more cameras located at an angle relative to the projector unit.

Color texture of the dental object may be acquired by illuminating the object using different monochromatic colors such as individual red, green and blue colors or my illuminating the object using multichromatic light such as white light. A 2D image may be acquired during a flash of white light.

Generally the process of obtaining surface information in real time of a dental object to be scanned requires the scanning device to illuminate the surface and acquire high number of 2D images. Typically a high speed camera is used with a framerate of 300-2000 2D frames pr second dependent on the technology and 2D image resolution. The high amount of image data needed to be handled by the scanning device to eighter directly forward the raw image data stream to an external processing device or performing some image processing before transmitting the data to an external device or display. This process requires that multiple electronic components inside the scanner is operating with a high workload thus requiring a high demand of current.

The scanning device comprises one or more light projectors configured to generate an illumination pattern to be projected on a three-dimensional dental object during a scanning session. The light projector(s) preferably comprises a light source, a mask having a spatial pattern, and one or more lenses such as collimation lenses or projection lenses. The light source may be configured to generate light of a single wavelength or a combination of wavelengths (mono-or polychromatic). The combination of wavelengths may be produced by using a light source configured to produce light (such as white light) comprising different wavelengths.

Alternatively, the light projector(s) may comprise multiple light sources such as LEDs individually producing light of different wavelengths (such as red, green, and blue) that may be combined to form light comprising the different wavelengths. Thus, the light produced by the light source may be defined by a wavelength defining a specific color, or a range of different wavelengths defining a combination of colors such as white light. In an embodiment, the scanning device comprises a light source configured for exciting fluorescent material of the teeth to obtain fluorescence data from the dental object. Such a light source may be configured to produce a narrow range of wavelengths. In another embodiment, the light from the light source is infrared (IR) light, which is capable of penetrating dental tissue. The light projector(s) may be DLP projectors using a micro mirror array for generating a time varying pattern, or a diffractive optical element (DOF), or back-lit mask projectors, wherein the light source is placed behind a mask having a spatial pattern, whereby the light projected on the surface of the dental object is patterned. The back-lit mask projector may comprise a collimation lens for collimating the light from the light source, said collimation lens being placed between the light source and the mask.

The mask may have a checkerboard pattern, such that the generated illumination pattern is a checkerboard pattern. Alternatively, the mask may feature other patterns such as lines or dots, etc.

The scanning device preferably further comprises optical components for directing the light from the light source to the surface of the dental object. The specific arrangement of the optical components depends on whether the scanning device is a focus scanning apparatus, a scanning device using triangulation, or any other type of scanning device. A focus scanning apparatus is further described in EP 2 442 720 B1 by the same applicant, which is incorporated herein in its entirety.

The light reflected from the dental object in response to the illumination of the dental object is directed, using optical components of the scanning device, towards the image sensor(s). The image sensor(s) are configured to generate a plurality of images based on the incoming light received from the illuminated dental object. The image sensor may be a high-speed image sensor such as an image sensor configured for acquiring images with exposures of less than 1/1000 second or frame rates in excess of 250 frames pr. second (fps). As an example, the image sensor may be a rolling shutter (CCD) or global shutter sensor (CMOS). The image sensor(s) may be a monochrome sensor including a color filter array such as a Bayer filter and/or additional filters that may be configured to substantially remove one or more color components from the reflected light and retain only the other non-removed components prior to conversion of the reflected light into an electrical signal. For example, such additional filters may be used to remove a certain part of a white light spectrum, such as a blue component, and retain only red and green components from a signal generated in response to exciting fluorescent material of the teeth.

The network unit may be configured to connect the dental scanning system to a network comprising a plurality of network elements including at least one network element configured to receive the processed data. The network unit may include a wireless network unit. The wireless network unit is configured to wirelessly connect the dental scanning system to the network comprising the plurality of network elements including the at least one network element configured to receive the processed data.

The dental scanning system preferably further comprises a processor configured to generate scan data (such as intraoral scan data) by processing the two-dimensional (2D) images acquired by the scanning device. The processor may be part of the scanning device. As an example, the processor may comprise a Field-programmable gate array (FPGA) and/or an Advanced RISC Machines (ARM) processor located on the scanning device. The scan data comprises information relating to the three-dimensional dental object. The scan data may comprise any of: 2D images, 3D point clouds, depth data, texture data, intensity data, color data, and/or combinations thereof. As an example, the scan data may comprise one or more point clouds, wherein each point cloud comprises a set of 3D points describing the three-dimensional dental object. As another example, the scan data may comprise images, each image comprising image data e.g. described by image coordinates and a timestamp (x, y, t), wherein depth information can be inferred from the timestamp. The image sensor(s) of the scanning device may acquire a plurality of raw 2D images of the dental object in response to illuminating said object using the one or more light projectors. The plurality of raw 2D images may also be referred to herein as a stack of 2D images. The 2D images may subsequently be provided as input to the processor, which processes the 2D images to generate scan data. The processing of the 2D images may comprise the step of determining which part of each of the 2D images are in focus in order to deduce/generate depth information from the images. The depth information may be used to generate 3D point clouds comprising a set of 3D points in space, e.g., described by cartesian coordinates (x, y, z). The 3D point clouds may be generated by the processor or by another processing unit. Each 2D/3D point may furthermore comprise a timestamp that indicates when the 2D/3D point was recorded, i.e., from which image in the stack of 2D images the point originates. The timestamp is correlated with the z-coordinate of the 3D points, i.e., the z-coordinate may be inferred from the timestamp.

Accordingly, the output of the processor is the scan data, and the scan data may comprise image data and/or depth data, e.g. described by image coordinates and a timestamp (x, y, t) or alternatively described as (x, y, z). The scanning device may be configured to transmit other types of data in addition to the scan data. Examples of data include 3D information, texture information such as infra-red (IR) images, fluorescence images, reflectance color images, x-ray images, and/or combinations thereof.

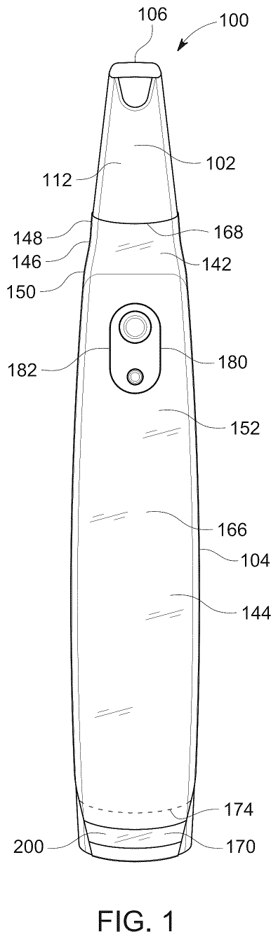

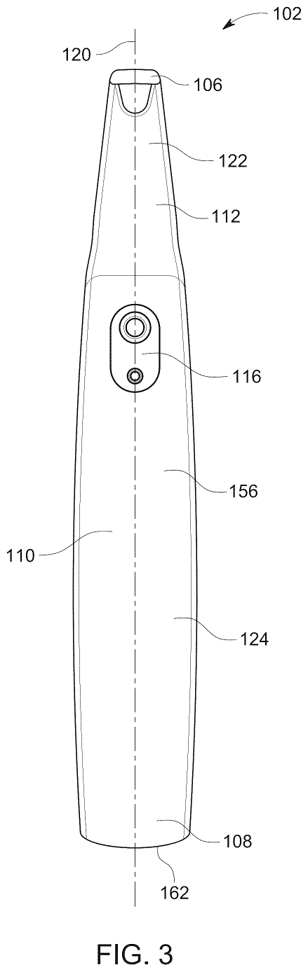

Referring to FIG. 1, a top perspective view of an intraoral scanner system 100 having an intraoral scanner 102 (hereinafter referred to as scanner 102) and a sleeve 104 arranged partially covering the scanner 102 is shown. The scanner 102 is configured to be inserted inside a mouth of a patient to perform scanning/imaging of a dental object. Accordingly, the scanner 102 is configured to acquire intraoral scan data from a three-dimensional dental object during a scanning session to prepare 3 dimensional images of the dental object. As shown in FIG. 3, the scanner 102 includes a front end 106, a rear end 108, a main body 110 extending from the rear end 108 towards the front end 106, and a protection tip 112 removably/detachably coupled to the main body 110 and extending from the main body 110 to the front end 106 of the scanner 102.

Further, the scanner 102 includes a user interface 116 defined at the main body 110 and arranged proximate to the protection tip 112.

The user interface 116 includes at least one button/switch adapted to be manipulated by a medical practitioner to operate the scanner 102 and perform the scan. Additionally, sides of the scanner 102 are tapered inwardly in a direction from the rear end 108 to the front end 106.

Accordingly, a cross-sectional area of the scanner 102 varies along a central longitudinal axis 120 of the scanner 102 and decreases in the direction of the longitudinal axis 120 from the rear end 108 towards the front end 106. As shown, a cross-sectional area of the scanner 102 at the rear end 108 is larger than a cross-sectional area of the scanner 102 at the front end 106. In the illustrated embodiment, the scanner 102 includes a first part 122 having a first cross-sectional area and a second part 124 having a second cross-sectional area. The second cross-sectional area is larger than the first cross-sectional area.

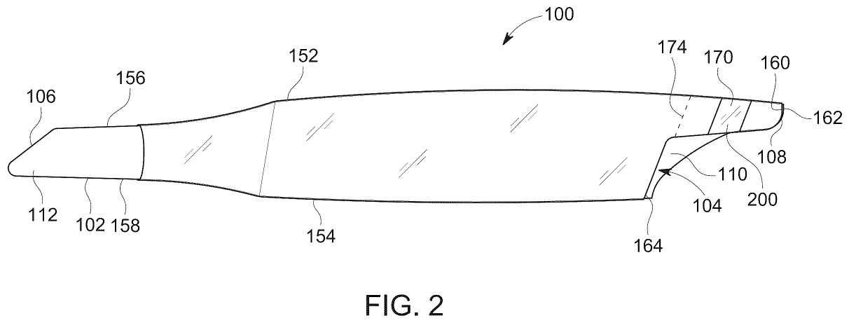

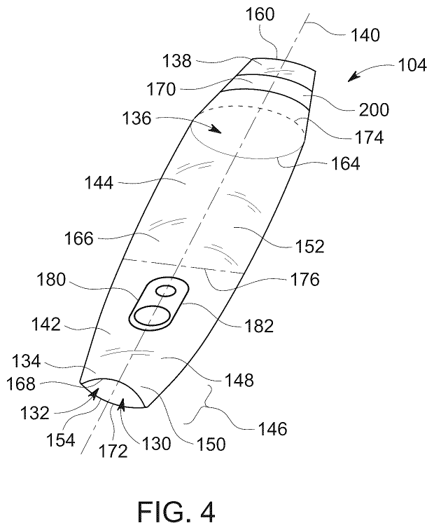

Referring to FIGS. 1, 2, 4, and 5, the sleeve 104 defines a space 130 (shown in FIG. 4) for receiving at least a portion of the main body 110 and a portion of the protection tip 112, and includes a first opening 132 arranged at a front end 134 (i.e., first end 134) of the sleeve 104 and a second opening 136 arranged proximate to a rear end 138 (i.e., second end) of the sleeve 104. To accommodate the main body 110 and a portion of the protection tip 112, a cross-sectional area of the sleeve 104 also varies along its longitudinal axis 140 such that the first opening 132 is smaller than the second opening 136. Also, the first opening 132 is smaller than a cross-sectional area of at least a part of the protection tip 112 of the scanner 102 and is larger than a cross-sectional area of a part of the protection tip 112. Accordingly, as shown in FIG. 1 and FIG. 2, the first opening 132 allows a part of the protection tip 112 to extend outside the sleeve 104 in the assembly of the sleeve 104 with the scanner 102. To facilitate a tight fit of the sleeve 104 over the scanner 102, at least a part of the sleeve 104 has a shape that corresponds to at least a part of an outer shape of the protection tip 112 and/or to at least a part of an outer shape of the main body 110 of the scanner 102.

Also, the sleeve 104 includes a first portion 142 extending from the front end 134 towards the rear end 138 and a second portion 144 extending from the first portion 142 to the rear end 138. As shown, the first portion 142 includes a cross-sectional area that is greater/larger than the cross-sectional area of the first portion 142. As can be seen from FIG. 1, the cross-sectional area of the first part 122 of the scanner 102 is smaller than the cross-sectional area of the second portion 144 of the sleeve 104, and the cross-sectional area of the second part 124 of the scanner is larger than the cross-sectional area of the first portion 142 of the sleeve 104. Accordingly, to assemble the sleeve 104 over the scanner 102, the first part 122 of the scanner 102 is inserted inside the sleeve 104 through the second opening 136, and pushed in a longitudinal direction such that a portion of the first part 122 of the scanner 102 extends outwardly of the first opening 132 of the sleeve 104, with the remaining of the first part 122 of the scanner 102 arranged inside the sleeve 104, and the second part 124 of the scanner 102 is arranged inside the second portion 144 of the sleeve 104. Additionally, the sleeve 104 has a transition area 146 that extends along the longitudinal axis 140 of the sleeve. At a first part 148 of the transition area 146, the cross-sectional area of the scanner 102 is equal to the cross-sectional area of the sleeve 104, and at a second part 150 of the transition area 146, the cross-sectional area of the scanner 102 is larger than the cross-sectional area of the sleeve 104 providing a sealing effect between the scanner 102 and the sleeve 104. Accordingly, the sleeve 104 is shaped and sized to provide hermetic sealing between the inner surface of the sleeve 104 and the outer surface of the scanner 102 (i.e., a portion of the protection tip 112 and/or a portion of the main body 110) to prevent fluid from passing through between the first opening 132 and at least a part of a surface of the protection tip 112 and/or at least a part of a surface of the main body 110.

Also, to facilitate an easy assembly of the sleeve 104 with the scanner 102 and to provide a tight fit and sealing effect between the sleeve 104 and the scanner 102 when the sleeve 104 is positioned on the scanner 102, the sleeve 104 may be made of a flexible material. In an embodiment, the sleeve 104 may be made of a polymer material. Also, in some embodiments, the sleeve 104 is made of a biomaterial or a biodegradable material.

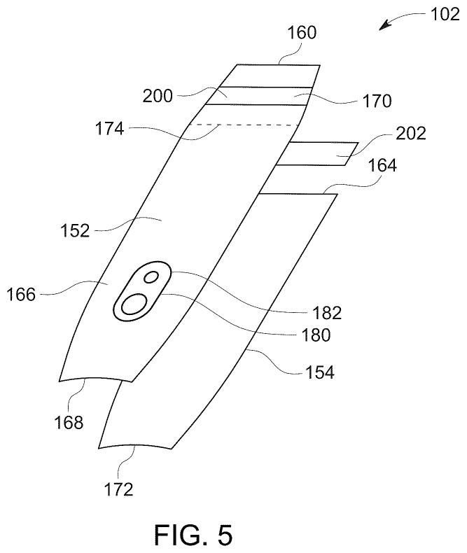

Further, as best shown in FIG. 4 and FIG. 5, the sleeve 104 includes a top part 152 and a bottom part 154 connected to each other along respective side edges, thereby defining the first opening 132 and the second opening 136 therebetween. As shown, the top part 152 is adapted to cover an upper portion 156 of the scanner 102 having the user interface 116, while the bottom part 154 covers a lower portion 158 of the scanner 102 arranged opposite to the upper portion 156. In the illustrated embodiment, the top part 152 covers the complete main body 110 and a portion of the protection tip 112 disposed proximate to the main body 110. As shown in FIG. 2, In the assembly of the sleeve 104 and the scanner 102, a rear edge 160 of the top part 152 of the sleeve 104 aligns with a rear edge 162 of the upper portion 156 of the scanner 102, while a rear edge 164 of the bottom part 154 of the sleeve 104 is arranged offset from the rear edge 160 of the upper portion 156 and arranged inwardly of the rear edge 160 of the top part 152. Accordingly, the top part 152 includes a first portion 166 extending from a front edge 168 of the top part 152 towards the rear edge 160 of the top part 152, and a flap portion 170 extending from the first portion 166 to the rear edge 160. As shown, a length of the first portion 166 is substantially equal to a length of the bottom part 154 of the sleeve 104, with the front edge 168 of the top part 152 arranged aligned with and underneath a front edge 172 of the bottom part 154. Accordingly, the front edge 168 of the top part 152 and the front edge 172 of the bottom part 154 are connected to each other and defines the first opening 132 of the sleeve 104.

Referring again to FIG. 4, as the length of the bottom part 154 is equal to the length of the first portion 166, the rear edge 164 of the bottom part 154 of the sleeve 104 aligns with and is arranged underneath a virtual line 174 separating the first portion 166 and the flap portion 170. Accordingly, the rear edge 164 of the bottom part 154 and the virtual line 174 of the top part 152 together defines the second opening 136. Therefore, the bottom part 154 of the sleeve 104 ends at the second opening 136, and the top part 152 of the sleeve 104 extends further from the second opening 136. Accordingly, as shown in FIGS. 2, 4, and 5., at the second end (i.e., rear end 138) of the sleeve 104, the top part 152 is longer than the bottom part 154 providing an air gap (shown in FIG. 2) between the scanner 102 and the sleeve 104 when the scanner 102 is arranged within the sleeve 104. In this manner, the second opening 136 allows airflow from the scanner 102 towards ambient air to remove heat from a battery compartment arranged at a rear of the scanner 102.

Moreover, the front edge 168 of the top part 152 is curved and includes a substantially concave shape. Similarly, the front edge 172 of the bottom part 154 is curved and includes a substantially concave shape. Accordingly, the first opening 132 is at least partly made from a curved cut. As shown, the front edges 168, 172 are curved in a transverse direction along a transverse axis 176 of the sleeve 104. Accordingly, a circumference of the first opening 132 is curved along the transverse axis 176 of the sleeve 104. Although concave front edges 168, 172 are shown and contemplated, it may be appreciated that the front edges 168, 172 may be convex. In some embodiments, a circumference of the first opening 132 is curved at least between the bottom part 154 and the top part 152.

Additionally, the sleeve 104 includes an indicator 180 arranged on the top part 152 of the sleeve 104 to guide a user about a method in which the scanner 102 needs to be accommodated/arranged/assembled inside the sleeve 104. The indicator 180 also facilitates in guiding the user in a direction of the assembly or insertion of the scanner 102 inside the sleeve 104, and therefore enables the user to correctly draw the sleeve 104 over the scanner 102. In an embodiment, the indicator 180 includes a visual marking 182 mimicking a shape of the user interface 116 of the scanner 102, thereby indicating the user that the visual marking 182 is to be arranged over the user interface 116 in the assembly of the sleeve 104 over the scanner 102. Accordingly, the visual marking 182 guides and ensures correct orientation and positioning of the sleeve 104 over the scanner 102. In some embodiments, the indicator 180 may include an uneven surface. The uneven surface may correspond to the raised and lowered portions of the user interface 116, and is adapted to align with the raised and lowered portions of the user interface 116 in the assembly of the sleeve 104 and the scanner 102.

To securely engage the sleeve 104 with the scanner 102 and to prevent sliding and disengagement of the sleeve 104 from the scanner 102, the sleeve 104 includes a layer 200 of adhesive material to adhesively engage/connect an inner surface of the sleeve 104 to an outer surface of the scanner 102. In an embodiment, the layer 200 of adhesive material is covered/protected by a tape 202 which is removed from the sleeve 104 before adhesively engaging the sleeve 104 with the scanner 102. The tape 202 covers the layer 200 of adhesive material and prevents deterring or damage of the adhesive material. In the illustrated embodiment, the tape 202 is removed after positioning the sleeve 104 over the scanner 102. As shown, the layer 200 of adhesive material is arranged proximate at the rear end 138 of the sleeve 104 and is disposed proximate to the rear edge 160 of the top part 152. As shown, the layer 200 of adhesive material is applied/arranged on an inner surface of the flap portion 170 of the top part 152.

Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

### ITEMS

1A. An intraoral scanner system comprising:
an intraoral scanner configured to acquire intraoral scan data from a three-dimensional dental object during a scanning session, and wherein the intraoral scanner includes;
   a protection tip;
   a main body, and wherein the protection tip is detachable to the main body; and
a sleeve configured to cover at least a part of the main body of the intraoral scanner.

1B. An intraoral scanner system comprising:
an intraoral scanner configured to acquire intraoral scan data from a three-dimensional dental object during a scanning session, and wherein the intraoral scanner includes;
   a main body, and wherein the protection tip is detachable to the main body; and
a sleeve configured to cover at least a user interface of the main body.

1C. An intraoral scanner system comprising:
an intraoral scanner configured to acquire intraoral scan data from a three-dimensional dental object during a scanning session, and wherein the intraoral scanner includes;
   a main body, and wherein the protection tip is detachable to the main body; and
a sleeve configured to cover at least a part of the main body, and wherein the at least part is configured to be handheld during the use of the intraoral scanner.

1D. An intraoral scanner system comprising:
an intraoral scanner configured to acquire intraoral scan data from a three-dimensional dental object during a scanning session, and wherein the intraoral scanner includes;
   a main body, and wherein the protection tip is detachable to the main body; and a sleeve configured to cover at least a part of the main body, and wherein the at least part is configured to be supported by a user during the use of the intraoral scanner.

1E. An intraoral scanner system, comprising a sleeve adapted to enclose at least a part of a handheld intraoral scanner or at least a part of a detachable protection tip of a handheld intraoral scanner.

2. An intraoral scanner system according to any of the previous items, wherein the sleeve comprises an indicator that is configured to guide how the intraoral scanner should be accommodated by the sleeve.

3. An intraoral scanner system according to any of the previous items, wherein the sleeve comprises an indicator that is configured to indicate that the sleeve is drawn correctly along the main body.

4. An intraoral scanner system according to item 2, wherein the indicator is a visual marking.

5. An intraoral scanner system according to item 2, wherein the indicator is an uneven surface.

6. An intraoral scanner system according to any of items 2-5, wherein the indicator is configured to mimic the shape of the user interface.

7. An intraoral scanner system according to any of the previous items, wherein the sleeve comprises a first end and a second end, wherein the second end is arranged opposite to the first end, and wherein a first opening of the sleeve is arranged at the first end and a second opening of the sleeve is arranged at the second end.

8. An intraoral scanner system according to the previous item 7, wherein the second opening is larger than the first opening.

9. An intraoral scanner system according to any of the previous items 7-8, wherein the first opening is at least partly made from a curved cut.

10. An intraoral scanner system according to any of the previous items 7-9, wherein a circumference of the first opening is curved along a transverse axis of the sleeve.

11. An intraoral scanner system according to any of the previous items 7-11, wherein the second opening is configured to allow airflow from a handheld intraoral scanner towards ambient air.

12. An intraoral scanner system according to any of the previous items, wherein at least a part of the sleeve has a shape that corresponds to at least a part of an outer shape of the protection tip or to at least a part of an outer shape of the main body of the intraoral scanner.

13. An intraoral scanner system according to any of the previous items, wherein the sleeve is configured to accommodate the intraoral scanner and configured to provide space for the intraoral scanner to slide into the sleeve.

14. An intraoral scanner system according to any of the previous items, wherein a cross-sectional area of the intraoral scanner varies along a longitudinal axis, and a cross-sectional area of the scanner is smaller than a cross-sectional area of the sleeve along a first part of the scanner along the longitudinal axis, and a cross-sectional area of the scanner is larger than a cross-sectional area of the sleeve along a second part of the scanner along the longitudinal axis.

15. An intraoral scanner system according to item 14, wherein the sleeve comprises a transition area along the longitudinal axis, wherein at a first part of the transition area, the cross-sectional area of the scanner is equal to the cross-sectional area of the sleeve, and wherein at a second part of the transition area, the cross-sectional area of the scanner is larger than the cross-sectional area of the sleeve providing a sealing effect between the scanner and sleeve.

16. An intraoral scanner system according to any of items 7-9, wherein the first opening is smaller than the cross-sectional area of at least a part of the intraoral scanner or the cross-sectional area of at least a part of the protection tip of the intraoral scanner.

17. An intraoral scanner system according to item 7, wherein the sleeve comprises a top part and a bottom part.

18. An intraoral scanner system according to the previous item 17, wherein the top part is arranged opposite to the bottom part.

19. An intraoral scanner system according to item 16, wherein when an intraoral scanner is arranged within the sleeve, the top part is arranged at a user interface of the scanner.

20. An intraoral scanner system according to item 7 and to any of items 17-19, wherein a circumference of the first opening is curved at least between the bottom part and the upper part.

21. An intraoral scanner system according to any of the items 17-20, wherein the top part of the sleeve is longer than the bottom part of the sleeve.

22. An intraoral scanner system according to the previous item 21, wherein at the second end the top part is longer than the bottom part providing an air gap between the intraoral scanner and the sleeve when the intraoral scanner is arranged within the sleeve.

23. An intraoral scanner system according to any of items 17-22, wherein a part of a first top distal end of the sleeve and a part of a first bottom distal end of the sleeve meet at the first end, and wherein at least a part of a second distal end the bottom part of the sleeve opposite to the first bottom distal end of the sleeve meets with at least a part of the top part of the sleeve which is not a distal end of the top part of the sleeve.

24. An intraoral scanner system according to item 17, wherein the bottom part of the sleeve ends at the second opening, and wherein the top part of the sleeve extends further from the second opening.

25. An intraoral scanner system according to items 3-6 and items 17-24, wherein the indicator is arranged on the top part of the sleeve.

26. An intraoral scanner system according to any of the previous items, wherein the sleeve is configured to provide hermetic sealing of a surface of at least a part of the intraoral scanner or of a surface of at least a part of a detachable tip of a handheld intraoral scanner.

27. An intraoral scanner system according to any of the previous items, wherein the sleeve is configured to seal at least a part of a surface of the protection tip and/or at least a part of the main body.

28. An intraoral scanner system according to item 7, wherein the sleeve is configured to prevent fluid from passing through between the first opening and at least a part of a surface of the protection tip and/or at least a part of a surface of the main body.

29. An intraoral scanner system according to any of the previous items, wherein the sleeve is at least partly made of a flexible material.

30. An intraoral scanner system according to any of the previous items, wherein the sleeve is at least partly made of a polymer material.

31. An intraoral scanner system according to any of the previous items, wherein the sleeve is at least partly made of a biomaterial or a biodegradable material.

32. An intraoral scanner system according to any of the previous items, wherein the sleeve comprises an adhesive material, configured to provide an adhesive connection between the sleeve and the intraoral scanner.

33. An intraoral scanner system according to the previous item 32, wherein the adhesive material is covered by a tape, configured to cover the adhesive material and configured to prevent deterring or damage of the adhesive material.

34. An intraoral scanner system according to the previous item and item 23, wherein the adhesive material is provided at the second end or at a second top distal end of the sleeve opposite to the first top distal end of the sleeve.

35. An intraoral scanner system according to any of the items 17-31 and 32-34, wherein the adhesive is arranged at the top part of the sleeve, and closer to the second end than to the first end.

1. An intraoral scanner system comprising:
an intraoral scanner configured to acquire intraoral scan data from a three-dimensional dental object during a scanning session, and wherein the intraoral scanner includes;
a protection tip;
a main body, and wherein the protection tip is detachable to the main body; and
a sleeve configured to cover at least a part of the main body of the intraoral scanner, and wherein the sleeve comprises a shape that follows at least a part of an outer surface of the protection tip, and the main body such that the shape of the sleeve is configured to tighten around at least a part of the protection tip when the handheld intraoral scanner is slided into the sleeve, wherein the sleeve comprises an indicator on a top part of the sleeve that is configured to visually guide a user on how the intraoral scanner should be accommodated by the sleeve, such that the top part and a bottom part of the sleeve are aligned to a top part and a bottom part of the main body, respectively.

2. (canceled)

3. An intraoral scanner system according to claim 1, wherein the sleeve comprises an indicator that is configured to indicate that the sleeve is drawn correctly along the main body, wherein the indicator is a visual marking, and the indicator is an uneven surface comprising an elevated part.

4. An intraoral scanner system according to claim 1, wherein the indicator is configured to mimic the shape of a user interface of the handheld intraoral scanner.

5. An intraoral scanner system according to claim 1, wherein the sleeve comprises a first end and a second end, wherein the second end is arranged opposite to the first end, and wherein a first opening of the sleeve is arranged at the first end and a second opening of the sleeve is arranged at the second end, and wherein the second opening is larger than the first opening.

6. An intraoral scanner system according to claim 5, wherein a circumference of the first opening is curved along a transverse axis of the sleeve.

7. An intraoral scanner system according to claim 5, wherein at the second end of the sleeve the top part is longer than the bottom part forming a second opening that provides an air gap between the intraoral scanner and the sleeve when the intraoral scanner is arranged within the sleeve.

8. An intraoral scanner system according to claim 1, wherein at least a part of the sleeve has a shape that corresponds to at least a part of an outer shape of the protection tip or to at least a part of an outer shape of the main body of the intraoral scanner.

9. An intraoral scanner system according to claim 1, wherein the sleeve is configured to accommodate the intraoral scanner and configured to provide space for the intraoral scanner to slide into the sleeve.

10. An intraoral scanner system according to claim 1, wherein a cross-sectional area of the intraoral scanner varies along a longitudinal axis, and a cross-sectional area of the scanner is smaller than a cross-sectional area of the sleeve along a first part of the scanner along the longitudinal axis, and a cross-sectional area of the scanner is larger than a cross-sectional area of the sleeve along a second part of the scanner along the longitudinal axis.

11. An intraoral scanner system according to claim 10, wherein the sleeve comprises a transition area along the longitudinal axis, wherein at a first part of the transition area, the cross-sectional area of the scanner is equal to the cross-sectional area of the sleeve, and wherein at a second part of the transition area, the cross-sectional area of the scanner is larger than the cross-sectional area of the sleeve providing a sealing effect between the scanner and sleeve.

12. An intraoral scanner system according to claim 5, wherein the first opening is smaller than the cross-sectional area of at least a part of the intraoral scanner or the cross-sectional area of at least a part of the protection tip of the intraoral scanner.

13. An intraoral scanner system according to claim 5, wherein the bottom part of the sleeve comprises a first bottom distal end and a second bottom distal end opposite to the first bottom distal end, and wherein the top part of the sleeve comprises a first top distal end and a second top distal end, and wherein the top part of the sleeve is longer than the bottom part of the sleeve, and wherein the second opening is provided by a part of the second bottom distal end that meets with a part of the top part of the sleeve located between the first top distal end and the second top distal end, and wherein the second opening is configured to allow air flow to and from the intraoral scanner.

14. An intraoral scanner system according to claim 1, wherein the sleeve is at least partly made of a first material that is more flexible than a second material of the main body of the intraoral scanner.

15. An intraoral scanner system according to claim 5, wherein the sleeve comprises an adhesive material, configured to provide an adhesive connection between the sleeve and the intraoral scanner, wherein the adhesive material is covered by a tape, configured to cover the adhesive material and configured to prevent deterring or damage of the adhesive material, and wherein the adhesive material is arranged at the top part of the sleeve closer to the second end of the sleeve than to the first end of the sleeve.
